(19) United States
(12) Patent Application Publication
(10) Pub. No.: US 2011/0264004 A1
(43) Pub. Date: Oct. 27, 2011

(54) Bronchoscopy System

(76) Inventor: Kenneth L. Willeford, Little River, SC (US)

(21) Appl. No.: 12/768,188

(22) Filed: Apr. 27, 2010

(51) Int. Cl. A61B 1/267; A61B 10/04; A61M 16/00

(52) U.S. Cl. 600/562; 128/207.14; 600/104

(57) ABSTRACT

A bronchoscopy oxygenation system may be used to permit the introduction of oxygen (or other gaseous substance) through a bronchoscopy device while also permitting various procedures, for example, biopsy, lavage, and/or suction.

### FIELD OF THE INVENTION

Various embodiments of the invention may find applicability in the field of pulmonary medicine: for example, in patient diagnosis, aspirating, sampling and therapeutic delivery.

### BACKGROUND OF THE INVENTION

Patients who have pulmonary (lung) symptoms may be candidates for bronchoscopies. There are two types of bronchoscopies; therapeutic and diagnostic. Therapeutic bronchoscopy may be used to provide a treatment or therapy, and diagnostic bronchoscopy may be used to aid in the diagnosis of an underlying condition. The bronchoscope may be attached to a light source and advanced through the nares or mouth of the patient. Some bronchoscopes may allow the pulmonologist to view the placement of the tip of the bronchoscope on a monitor from a video chip, and other models may have an eye piece for viewing. Local anesthetics are often administered through the bronchoscope as it is advanced through the posterior pharynx and into the lungs. With direct visualization and manipulation of the tip with directional control, the vocal cords may be identified and the instrument advanced into the trachea. Because of protective airway reflexes and the unpleasant nature of the procedure, the patient is likely to have received sedative medications that will decrease the respiratory drive. In such circumstances, the respiratory rate will be decreased and the depth will be shallow. Consequently, oxygen delivery to the patient is reduced.

Once the bronchoscope is within the pulmonary system, it may be intermittently advanced and withdrawn with directional control to access desired portions of the patient's lung. Diagnostic modalities afforded by this procedure may include visualization of the trachea, bronchi, and bronchioles for identification of abnormal tissue or secretions, as well as the ability to obtain biopsy specimens for laboratory analysis of tissue identification. Biopsy specimens may be obtained by passing a biopsy forceps through the channel and excising a piece of tissue. Fluoroscopic x-ray guidance can be used intermittently throughout this procedure to aid in confirmation of the placement of the tip of the instrument.

A problem that may arise is the problem of hypoxemia in patients who are having bronchoscopies. Hypoxemia is defined as reduced levels of oxygen in the blood and can be determined, measured, and quantified by pulse oximitry. Pulse oximitry is a standard monitor that measures the saturation of the oxygen carrying hemoglobin molecule. In view of the problem of hypoxemia, it would be desirable to have a bronchial oxygenating system which is easy and safe to use; and which, when used along with a bronchoscope, will provide oxygen through a channel of the bronchoscope.

### PATENT LITERATURE

Lorenzen (U.S. Pat. No. 5,735,271) teaches a closed ventilation system apparatus which allows multiple access to the respiratory system through one or more access ports to ventilate the lungs with a gas or gases; to aspirate, oxygenate and visually inspect the respiratory system and/or take tissue samples.

Bayron (U.S. Pat. No. 5,746,199) teaches a device with an endotracheal tube having attached thereto having several entry ports.

Urrutia (U.S. Pat. No. 5,817,068) teaches a plurality of feeds to a main conduit. Wood (U.S. Pat. No. 5,766,211) is for a device with a canal with a three-way valve for feed into the canal.

Akiba (U.S. Pat. No. 6,425,535) is for a fluid supplying apparatus for a cleaning the observation window of an endoscope.

Socaria (U.S. Pat. No. 5,329,921) discloses an endotracheal device allowing for the performance of various medical procedures while maintaining continuity of respiration.

Willeford (U.S. Pat. No. 7,320,324) discloses a bronchoscopy system that permits the introduction of oxygen. The system of Willeford includes a pressure relief vent and/or pressure relief valve, as well as a stopcock to permit various usage scenarios.

### SUMMARY OF EMBODIMENTS OF THE INVENTION

Various embodiments of the invention may address a bronchoscopy system and/or method that may permit the delivery of oxygen directly to the lungs of patients during therapeutic and diagnostic bronchoscopy, which may be helpful in order to reduce hypoxia or hypoxemia during a procedure.

### BRIEF DESCRIPTION OF THE DRAWINGS

FIG. 1 is a diagram of an embodiment of a bronchoscopy oxygenation system according to some embodiments of the invention.

### DESCRIPTION OF VARIOUS EMBODIMENTS OF THE INVENTION

Hypoxia during bronchoscopy may be derived from three main causes. First, during bronchoscopy, patients may often be given sedative medications which decrease their respiratory effort, so less oxygen is delivered to the lungs. Second, the size of the bronchoscope, which may be large compared to the size of the opening between the vocal cords, can create a mechanical obstruction impeding oxygen delivery. The third main cause of hypoxia is the elimination of air and oxygen from the lungs during suctioning performed to remove secretions and improve visualization as well as suctioning for bronchoalveolar lavage. These factors combine to place these patients at risk for complications related to depressed levels of oxygen in their blood.

The bronchoscopy oxygenation system according to various embodiments of the present invention may be particularly useful in situations in which the patient is susceptible to receiving reduced oxygen during a procedure. Medical procedures in which the system can be used may include, for example:

a) Bronchoalveolar lavage, which is a technique that can be diagnostic and/or therapeutic in nature. In this process, fluid is administered through a channel into the lung airways and then recovered and collected with the use of suction, which may generally be attached to the channel of the bronchoscope. This process can be of diagnostic value as the fluid will contain cells from the patient's lung, which can be analyzed in the laboratory for tissue identification. In addition, bronchoalveolar lavage can be a therapeutic technique by removing excess and harmful secretions that may be found in the bronchoalveolar system. Improved flow and respiratory gas exchange can result following this technique.
b) Obtaining of tissue specimens. Biopsy specimens may be obtained by passing a biopsy forceps through the channel and excising a piece of tissue. Fluoroscopic x-ray guidance can be used intermittently during this procedure to aid in confirmation of the placement of the tip of the instrument.
c) Suctioning to remove fluids for laboratory analysis and/or to remove secretions that interfere with visualization. Suctioning may be performed to obtain fluid and secretions, as may be determined by the needs of diagnostic modalities or treatment options. In addition, suctioning may be intermittently and frequently performed throughout a procedure to facilitate visualization by the pulmonologist, as excess secretions within the lung can obscure visualization during the procedure. This suctioning, however, may also actively remove oxygen from within the lung, further leading to reduced oxygen delivery to the respiratory gas exchange membranes and resulting in hypoxemia.

In using the bronchoscopy oxygenation system of the invention along with a bronchoscope, the functions of the bronchoscope need not be impeded. The design of the bronchoscopy oxygenation system, according to various embodiments of the present invention, may utilize a single channel of a bronchoscope to provide oxygen. This channel may be used to obtain tissue biopsies by passing a biopsy forceps down the channel, may be used to administer fluids such as saline into the lungs to combine with the fluid and secretions in the lung for bronchoalveolar lavage, and/or may be used for suctioning to remove fluids for laboratory analysis or to remove secretions that interfere with visualization. Additionally, according to various embodiments of the invention, this channel may also be used for the purpose of administering oxygen.

The bronchoscopy oxygenation system according to various embodiments of the invention may be used with either spontaneous or controlled ventilation. Anesthetic systems have been classified as open, semi-open, semi-closed, or closed. Use of various embodiments of the inventive bronchoscopy oxygenation system is not limited by the configuration of the breathing system or the presence of controlled or spontaneous ventilation.

Embodiments of the present bronchoscopy oxygenation system according to embodiments of the invention may utilize a means that will allow for the safe administration of oxygen and still allow biopsies, lavage, or suctioning. The system may thus allow for a shared function of a single channel in a bronchoscope. Oxygen may be administered from a standard oxygen flow meter and delivered to a patient from the distal tip of the device.

The oxygen flow may be interrupted during some procedures in which instrumentation may block the channel. Interruption of oxygen flow may lead to a safety hazard, as the oxygen supply tubing may end up holding pressure equal to the oxygen outlet pressure, which may be, for example, 50 psi. When such a relatively high pressure is allowed to access a syringe, the plunger may become a forceful projectile, which may be of concern for personnel. If the pressure is allowed to pass through the bronchoscope, it may cause patient harm from barotrauma. This safety issue may be resolved, in various embodiments of the invention, by the use of a pressure relief vent that may be designed to reduce pressure (e.g., 50 psi) to a level that would be safe for patient application and/or a safety pressure relief valve; these may be built into the device according to various embodiments of the invention. The pressure relief valve may be set or adjusted, for example, at 40 cm of water, which may serve to protect a patient from a surge in pressure.

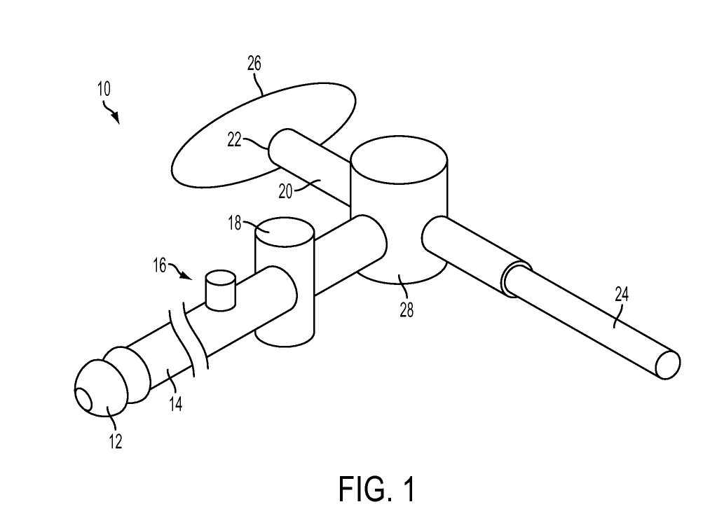

FIG. 1 shows a representation of a bronchoscopy oxygenation system according to an embodiment of the invention. The system 10 may have an oxygen entry orifice 12, which may be used to feed oxygen or another appropriate gaseous substance (e.g., but not limited to, air, mixed gases, etc.) to a feed channel 14. The feed channel may include a pressure relief vent 16, a pressure relief valve 18, or both; the pressure relief vent 16 and/or pressure relief valve 18 may be, for example, as described in U.S. Pat. No. 7,320,324, which is incorporated by reference herein. The feed channel 14 may be attached to an instrument insertion channel 20 having a proximal end 22 and a distal tip 24. The instrument insertion channel 20 may be configured to permit the introduction of a non-gaseous substance (such as an instrument or fluid) through the instrument insertion channel and into a patient. The proximal end 22 may be configured to receive a cover or cap 26; an example of such a part 26 may be design similar to a bronchoscopy biopsy valve, such as Part #MAJ 210, manufactured by Olympus, with dimensions appropriate for compatibility with proximal end 22 (i.e., scaled, if necessary). Other examples may include, but are not limited to, Tuohy-Borst adapters and other hemostasis valves. Such adapters or valves may be used to prevent backflow of fluid (gaseous or liquid) around an instrument inserted through a working channel of a medical device, such as a bronchoscope. Such a cover/cap 26 may need to be sufficiently airtight to withstand the pressure of a gas introduced through feed channel 14, for example, 40 cm of water; in the case of an adapter or valve that permits an instrument to be passed through it, this may need to be applicable whether or not an instrument is currently inserted. The outside diameter of the distal tip 24 may be designed to permit access to a bronchoscope biopsy valve and may have an inside diameter large enough to at least permit access by a bronchoalveolar lavage catheter and/or bronchoscope biopsy forceps. The instrument insertion channel 20 and the feed channel 14 may meet at a junction 28; junction 28 may, for example, be in the form of a “T” or any other appropriate form that does not block the flow of oxygen/gas from feed channel 14 into instrument insertion channel 20.

In operation, oxygen, or another gas, e.g., at 50 psi (which is the standard pressure used in many operating rooms in the United States; but note that the invention is not limited to use with a gas delivered at this pressure) may enter the device 10 through a tube that may be connected at entry orifice 12. The pressure relief vent 16 and/or pressure relief valve 18 may be appropriately sized to ensure that a pressure level of the oxygen/gas is limited to a level that is safe for delivery to a patient, e.g., 40 cm of water, and still allow an acceptable airflow, e.g., approximately 3 liters per minute, through the device. Oxygen/gas may then flow through the feed channel 14, to the junction 28, where it may then feed into the instrument insertion channel 20. When the cover/cap 26 is attached to the proximal end 22 of the instrument insertion channel 20, the oxygen/gas may then flow through the distal tip 24 (which may be inserted into the biopsy port of a bronchoscope (not shown)); i.e., the cover/cap 26 may prevent the oxygen/gas from flowing out the proximal end 22 of the instrument insertion channel 20 and thereby force the oxygen/gas to flow through the distal tip 24. The cover/cap 26 may be removed to permit the insertion of an instrument (not shown) into the proximal end 22 of instrument insertion channel 20, through the distal end 24, and into a patient. In alternative configurations, the cover/cap 26 may permit insertion of an instrument without removing cover/cap 26, such as with a bronchoscopy biopsy valve, Tuohy-Borst adapter, or hemostasis valve (any of which may be scaled, as necessary, to appropriate dimensions for compatibility with the proximal end 22 and/or with various types of instruments that may be used). Depending upon the size and configuration of the instrument, the instrument may partially or completely block the flow of oxygen/gas through the instrument insertion channel 20, for all or part of a period of time during which the instrument is inserted.

The bronchoscopy oxygenation system 10 may be used by inserting the device 10 into the biopsy port of a bronchoscope or some other compatible access orifice. Tubing may then be connected, e.g., from an oxygen flow meter to the device at orifice 12. In one example of use, the flow meter may be adjusted to approximately 10 liters of oxygen per minute; however, the inventive concepts are not limited to this example, and it is noted that embodiments of the present device may also be effective at much lower flow rates (it is noted that average oxygen consumption for an adult is 250 cc/minute, and embodiments of the invention may adequately supply oxygen at this rate). Cover/cap 26 may be initially attached to the proximal end 22 of the instrument insertion channel 20. When bronchoalveolar lavage is performed or tissue biopsies are obtained, for example, the cover/cap 26 may be removed to permit insertion of an appropriate instrument, and once the lavage or biopsies are completed, the cover/cap 26 may be replaced. Again, in alternative configurations, cover/cap 26 may permit insertion of an instrument without removing cover/cap 26, and in such configurations, the cover/cap 26 may reseal itself after removal of an instrument and/or completion of the lavage or biopsies.

Various embodiments of the system may be used during many pulmonary medicine procedures involving the lungs or bronchi where there is reduced oxygen at the respiratory gas exchange membranes in patients having bronchoscopies. The bronchoscopy oxygenation system of embodiments of the invention may address this problem by administering oxygen (or other appropriate gas) through the channel directly into the lungs. This oxygen delivery will be independent of reduced patient respiratory drive from intravenous medications, and also independent of the mechanical obstruction at the vocal cords created by the bronchoscope.

Obviously, many modifications may be made without departing from the basic spirit of the present invention. Accordingly, it will be appreciated by those skilled in the art that within the scope of the appended claims, the invention may be practiced other than has been specifically described herein.

Furthermore, benefits, other advantages, and solutions to problems may have been described above with regard to specific embodiments of the present invention. However, the benefits, advantages, solutions to problems, and any element(s) that may cause or result in such benefits, advantages, or solutions to become/becoming more pronounced are not to be construed as a critical, required, or essential feature(s) or element(s) of any or all of the claims. As used herein and in the appended claims, the terms, “comprises,” “comprising,” or any other variation thereof is intended to refer to a non-exclusive inclusion, such that a process, method, apparatus, or article of manufacture that comprises a list of elements is not limited to only the listed elements, but may include other elements not expressly listed or inherent to such process, method, apparatus, or article of manufacture.

What is claimed is:

1. A bronchoscopy system comprising:
an orifice for receiving a gaseous substance;
a gaseous substance feed channel coupled to the orifice to receive and feed the gaseous substance; and
an instrument insertion channel coupled to the gaseous substance feed channel to permit flow of the gaseous substance from the gaseous substance feed channel into the instrument insertion channel;
wherein the instrument insertion channel is configured to enable the gaseous substance to flow into the patient and to permit the insertion of a non-gaseous substance into the patient through the instrument insertion channel.

2. The bronchoscopy system of claim 1, wherein the feed channel comprises a pressure relief vent situated between the orifice and the instrument insertion channel.

3. The bronchoscopy system of claim 1, wherein the feed channel comprises a pressure relief valve situated between the orifice and the instrument insertion channel.

4. The bronchoscopy system of claim 1, further comprising a cap configured to be attached at a proximal end of the instrument insertion channel to prevent flow of the gaseous substance from the proximal end of the instrument insertion channel.

5. The bronchoscopy system of claim 4, wherein the cap is configured to be removed to permit insertion of the non-gaseous substance into the instrument insertion channel through the proximal end.

6. The bronchoscopy system of claim 5, wherein the non-gaseous substance comprises at least one item selected from the group consisting of an instrument and a fluid.

7. The bronchoscopy system of claim 6, wherein the non-gaseous substance comprises at least one instrument selected from the group consisting of: a bronchoalveolar lavage catheter, a bronchoscope biopsy forceps, and a suctioning instrument.

8. The bronchoscopy system of claim 4, wherein the cap is configured to allow the non-gaseous substance to be sealably inserted into the instrument insertion channel through the proximal end.

9. The bronchoscopy system of claim 8, wherein the non-gaseous substance comprises at least one item selected from the group consisting of an instrument and a fluid.

10. The bronchoscopy system of claim 9, wherein the non-gaseous substance comprises at least one instrument selected from the group consisting of: a bronchoalveolar lavage catheter, a bronchoscope biopsy forceps, and a suctioning instrument.

11. The bronchoscopy system of claim 4, wherein the cap comprises at least one item selected from the group consisting of a biopsy valve, a Tuohy-Borst adapter, and a hemostasis valve.

12. The bronchoscopy system of claim 1, wherein a distal end of the instrument insertion channel is configured to be inserted into a bronchoscope biopsy valve.

13. The bronchoscopy system of claim 1, further comprising a junction between the feed channel and the instrument insertion channel.

14. The bronchoscopy system of claim 13, wherein the junction comprises a “T” junction.
